(19) United States
(12) Patent Application Publication
(10) Pub. No.: US 2016/0096928 A1
(43) Pub. Date: Apr. 7, 2016

(54) POLYIMIDES, PROCESSES FOR PRODUCING SAID POLYIMIDES AND ARTICLES OBTAINED FROM SAID POLYIMIDES

(71) Applicant: RHODIA OPERATIONS, Paris (FR)

(72) Inventor: Stephane JEOL, Saint-Fons (FR)

(21) Appl. No.: 14/785,884

(22) PCT Filed: Apr. 22, 2014
(86) PCT No.: PCT/EP2014/058100

§ 371 Date: Oct. 21, 2015

(30) Foreign Application Priority Data
Apr. 24, 2013 (FR) 1353729

(51) Int. Cl. C08G 73/10; C07C 53/00; C07D 307/54

(52) U.S. Cl. CPC C08G 73/1067; C08G 73/1007; C07D 307/54; C07C 53/00

(57) ABSTRACT

The present invention relates to (co)polymers comprising at least one imide function, making use of at least one diamine chosen from 2,5-bis(aminomethyl)furan and 2,5-bis(aminomethyl)tetrahydrofuran, or diisocyanate equivalents thereof. These (co)polymers can be converted into articles by various methods.

The present invention relates to novel (co)polymers and to the processes for the synthesis thereof. More specifically, the present invention is directed toward (co)polymers comprising at least one imide function using a particular diamine or a diisocyanate derivative thereof. These (co)polymers can be converted into articles by various methods.

### PRIOR ART

Polyimides, in particular aromatic polyimides, are known for their exceptional thermal and/or mechanical properties, which mark them out in particular for “high-performance” applications in various fields such as aeronautics or else electronics (printed circuit boards for example). They are generally infusible, that is to say they decompose before melting (at more than 500° C.), and are considered to be thermosets, that is to say, once formed, they cannot be processed by remelting. They are generally characterized by excellent mechanical strengths and low thermal expansion coefficients owing to very high glass transition temperatures (Tg), generally above 200° C., or even above 250° C.

These aromatic polyimides are generally synthesized according to a two-step process: synthesis of a polyamic acid from an aromatic dianhydride and an aromatic diamine, then cyclization to polyimide during the production of the articles. However, these aromatic polyimides have a certain number of drawbacks associated with the use of aromatic diamines, most of which are carcinogenic. Indeed, the toxicity of these diamines imposes very careful handling conditions during the synthesis of polyamic acids and also care in controlling the amount of aromatic diamine free in the polyamic acid solutions before their use and on the final polyimide.

In order to reduce the risks associated with the handling of these diamines, an alternative consists in partially or totally substituting the aromatic diamines (the amine function is directly bonded by a carbon atom which is part of an aromatic ring) with aliphatic, cycloaliphatic or arylaliphatic diamines, the amine functions of which are bonded to a carbon atom which is not part of an aromatic ring. In U.S. Pat. No. 7,691,471, particular cycloaliphatic diamines which make it possible to obtain polyimides with high Tgs, for example above 250° C., are described. U.S. Pat. No. 3,179,614 describes, for its part, polyamides-precursor polyamic acids obtained by reaction between pyromellitic anhydride and meta-xylylenediamine(arylaliphatic diamine) or else between pyromellitic anhydride and 2,11-dodecanediamine(aliphatic diamine). However, the polyimides obtained with aliphatic diamines have a low glass transition temperature, below 200° C., as described in U.S. Pat. No. 2,710,853, thus limiting the performances of these polymers and therefore their potential in “high-performance” applications.

Moreover, polymers synthesized from biobased monomers are of major interest nowadays since they make it possible to reduce the environmental footprint. There are a large number of combinations of biobased monomers or of combinations of biobased monomers and of monomers resulting from fossil resources which can be used to generate polymers, which are then said to be “biobased”. Some of these biobased polymers can be used to replace polymers resulting from fossil resources.

The term “biobased” is intended to mean that it concerns a material derived from renewable resources. A renewable resource is a animal or plant natural resource, the stock of which can be reconstituted over a short period on the human scale. It is in particular necessary for this stock to be able to be renewed as quickly as it is consumed.

Unlike materials resulting from fossil materials, renewable raw materials contain a high proportion of ¹⁴C. This characteristic can in particular be determined via one of the methods described in standard ASTM D6866, in particular according to the mass spectrometry method or the liquid scintillation spectrometry method.

These renewable resources are generally produced from cultivated or non-cultivated plant matter, such as trees, plants, for example sugarcane, corn, cassava, wheat, rape, sunflower, palm, castor oil plant or the like, or from animal matter, such as fats (tallow, and the like).

Among the biobased monomers, there is great interest in molecules bearing a furan ring, in particular 2,5-furandicarboxylic acid derivatives, obtained, for example, from hydroxymethylfurfural (HMF), which is itself obtained, for example, from sugars, such as glucose.

In the biobased polyimide field, the article “Polyimides based on furanic diamines and aromatic dianhydrides: synthesis, characterization and properties”, Polym (2011) 67:1111-1122, describes polyimides obtained by polycondensation between aromatic dianhydrides and difuran diamines. However, the polyimides obtained both are amorphous and have a very low Tg (maximum of 143° C.). These polymers could therefore be used only at temperatures below 150° C., thereby ruling out the “high-performance” applications in which conventional polyimides are generally involved and applications as substitution for amorphous polymers with high glass transition temperatures, such as the polyetherimides known under the name Ultem® (Tg of approximately 220° C.), sold by Sabic, or else the polyamideimide Torlon® from Solvay (Tg of approximately 275° C.).

The objective of the present invention is therefore to find novel polymers which meet the requirements of “high-performance” applications, that is to say having good thermal, mechanical and dielectric (insulating) properties, and also good dimensional stability. Furthermore, the polymers will have to have a high Tg. In addition, it will be possible for the polymers to be obtained from a large variety of monomers, which exhibit low, or even zero, toxicity, and which are eco-friendly, biobased, inexpensive, and widely available and/or easy to synthesize.

### INVENTION

Particular (co)polymers, which completely or partly satisfy the abovementioned objectives, have just been demonstrated by the applicant.

The present invention thus relates to a (co)polymer comprising the recurring unit of formula I below:

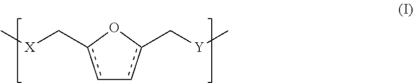

in which
X is a divalent group of formula Ia or Ib below:

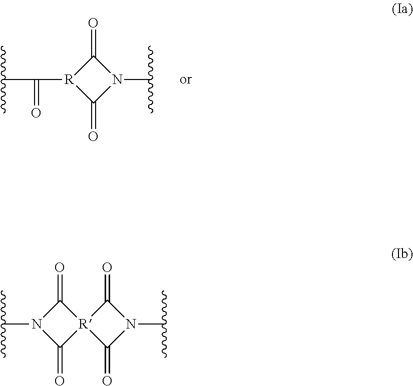

in which
R is a trivalent hydrocarbon-based group selected from:
   a saturated or unsaturated, aliphatic or cycloaliphatic group, optionally interrupted with one or more heteroatoms, and optionally substituted with one or more functional or non-functional groups;
   an aromatic, alkylaromatic or arylaliphatic group, optionally interrupted with one or more heteroatoms, and optionally substituted with one or more functional or non-functional groups;
R′ is a tetravalent hydrocarbon-based group selected from:
   a saturated or unsaturated, aliphatic or cycloaliphatic group, optionally interrupted with one or more heteroatoms, and optionally substituted with one or more functional or non-functional groups;
   an aromatic, alkylaromatic or arylaliphatic group, optionally interrupted with one or more heteroatoms, and optionally substituted with one or more functional or non-functional groups;
Y is:
   a covalent bond, when X corresponds to formula Ib,
   a divalent group of formula Ic or Id below, when X corresponds to formula Ia:

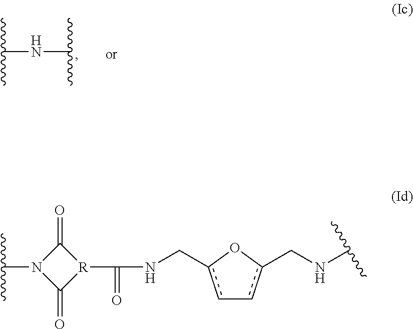

with R as defined above.

The (co)polymer according to the invention therefore comprises at least one imide function in its recurring unit of formula (I).

These (co)polymers can be prepared using, as constituent monomers, specific diamines or diisocyanates described hereinafter.

According to a first embodiment, the (co)polymer according to the invention can therefore be obtained by polymerization of the following compounds:
at least one compound A comprising two anhydride functions and/or its tetracarboxylic acid and/or ester equivalents, and
   at least one diamine B of formula II or III,

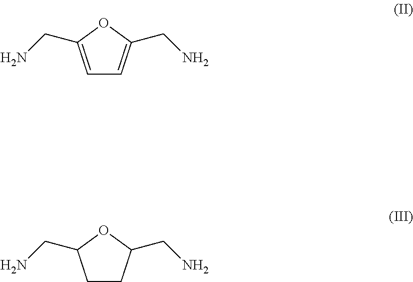 [SMILES: NCc1ccc(CN)o1 ; NCC1CCC(CN)O1]

or
   at least one diisocyanate B′ of formula II′ or III′ below:

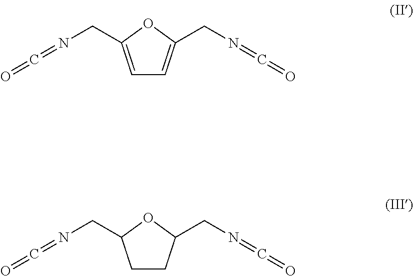 [SMILES: O=C=NCc1ccc(CN=C=O)o1 ; O=C=NCC1CCC(CN=C=O)O1]

or
at least one ammonium carboxylate salt obtained from the compounds A and B.

According to a second embodiment, the (co)polymer according to the invention can be obtained by polymerization of the following compounds:
at least one compound D comprising an anhydride function and a carboxylic acid function and/or its tricarboxylic acid and/or ester and/or acid chloride anhydride equivalents; and
   at least one diamine B of formula II or III,

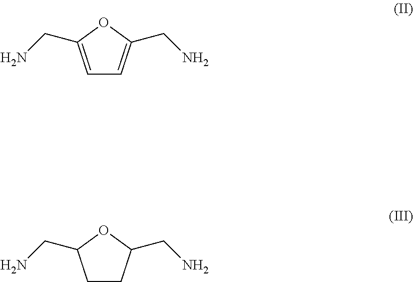 [SMILES: NCc1ccc(CN)o1 ; NCC1CCC(CN)O1]

or
   at least one diisocyanate B′ of formula II′ or III′ below:

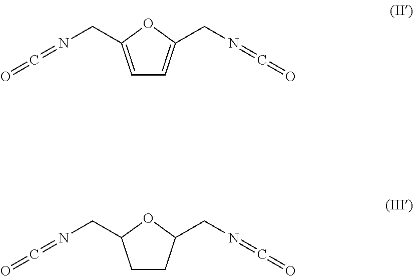 [SMILES: O=C=NCc1ccc(CN=C=O)o1 ; O=C=NCC1CCC(CN=C=O)O1]

or
at least one ammonium carboxylate salt obtained from the compounds D and B.

The (co)polymer of the invention can also be obtained by polymerization between at least the compound A and at least the diamine B or at least the diisocyanate B′ in the presence of:
when the diamine B is used: at least one diamine C of formula IV below:

NH₂—R₃—NH₂ (IV)
or
when the diisocyanate B′ is used: at least one diisocyanate C′ of formula IV′ below:

O═C═N—R₃—N═C═O (IV′)
with R₃ being a hydrocarbon-based divalent radical which is aliphatic, cycloaliphatic or arylaliphatic, and saturated and/or unsaturated, aromatic or alkylaromatic, and which optionally comprises heteroatoms.

The (co)polymer of the invention can also be obtained by polymerization between at least the compound D and at least the diamine B or at least the diisocyanate B′ in the presence of:
when the diamine B is used: at least one diamine C of formula IV below:

NH₂—R₃—NH₂ (IV)
or
when the diisocyanate B′ is used: at least one diisocyanate C′ of formula IV′ below:

O═C═N—R₃—N═C═O (IV′)
with R₃ being a hydrocarbon-based divalent radical which is aliphatic, cycloaliphatic or arylaliphatic, and saturated and/or unsaturated, aromatic or alkylaromatic, and which optionally comprises heteroatoms.

The present invention also relates to an ammonium carboxylate salt obtained from one or more compounds A and one or more diamines B and optionally one or more diamines C, said compounds A, B and C being as defined above or below in the description.

The present invention also relates to an ammonium carboxylate salt obtained from one or more compounds D and one or more diamines B and optionally one or more diamines C, said compounds D, B and C being as defined above or below in the description.

The invention also relates to processes for preparing the (co)polymers according to the invention by polymerization between at least one compound A and at least one diamine B or at least one diisocyanate B′, optionally in the presence of at least one diamine C when the diamine B is used, or of at least one diisocyanate C′ when the diisocyanate B′ is used, said compounds A, B, B′, C and C′ being as defined above or below in the description.

The invention also relates to processes for preparing the (co)polymers according to the invention by polymerization between at least one compound D and at least one diamine B or at least one diisocyanate B′, optionally in the presence of at least one diamine C when the diamine B is used, or of at least one diisocyanate C′ when the diisocyanate B′ is used, said compounds D, B, B′, C and C′ being as defined above or below in the description.

The invention also relates to compositions comprising the (co)polymer of the invention and fillers and/or additives.

The invention also relates to processes for producing articles based on a (co)polymer according to the invention, in particular:
by shaping by extrusion, molding or blow molding of said (co)polymer in molten form when it has a melting point below 350° C. for a semi-crystalline (co)polymer or a glass transition temperature below 300° C. for an amorphous (co)polymer, or
by cyclization of a polyamic acid intermediate as defined below in solid form or in the form of a solution or a dispersion in a solvent, said polyamic acid intermediate being advantageously obtained by reaction between the compounds A and B, optionally in the presence of diamine C.

The invention also relates to a polyamic acid, in particular obtained by reaction between the compounds A and B, optionally in the presence of diamine C, of formula V below:

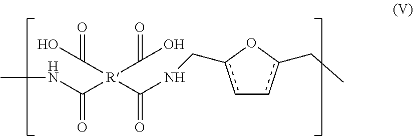

with R′ being as previously defined.

Finally, the invention therefore relates to an article obtained from the (co)polymer according to the invention or from the composition comprising same. It may be a molded part, for instance an injection-molded part or a composite comprising continuous fibers, an extruded part, for example a film, a fiber, a yarn or a filament, or else a blow-molded part. It may also be a foam. It may also be a part woven or knitted from fibers, yarns or filaments based on (co)polymer according to the invention

### DEFINITIONS

The term “semi-crystalline” is intended to mean a (co)polymer having an amorphous phase and a crystalline phase, for example having a degree of crystallinity of between 1% and 85%. The term “amorphous” is intended to mean a (co)polymer that does not have a crystalline phase detected by thermal analyses (of DSC “differential scanning calorimetry” type) and by x-ray diffraction.

The term “thermoplastic (co)polymer” is intended to mean a (co)polymer having a temperature above which the material softens and melts without degrading, and below which it becomes hard.

When a (co)polymer is prepared from an ammonium carboxylate salt, the melting temperature of the salt is preferably determined by measuring the endothermic endpoint temperature as measured by differential scanning calorimetry (DSC), using a Perkin Elmer Pyris 1 instrument, by heating the salt starting from 20° C. at a rate of 10° C./min.

The melting temperature of the (co)polymer is preferably determined at the peak of the fusion endotherm as measured by differential scanning calorimetry (DSC), using a Perkin Elmer Pyris 1 instrument, by heating the (co)polymer starting from 20° C. at a rate of 10° C./min. The determination of the glass transition temperature of the (co)polymer is taken at the average point using the tangent method by heating the (co)polymer starting from 20° C. at a rate of 10° C./min.

The (co)polymer according to the invention comprises the recurring unit I, that is to say it comprises at least one imide function in its recurring unit I.

In the case where the (co)polymer of the invention comprises at least one imide function and at least one amide function in its recurring unit I (that is to say when X=Ia and Y=Ic or Id), the term then used is (co)polyamideimide.

In the case where the (co)polymer of the invention comprises two imide functions in its recurring unit I (that is to say when X=Ib and Y is a covalent bond), the term used is (co)polyimide.

The (co)polymer according to the invention can consist essentially of the recurring unit of formula I. It is then a homopolymer. The term “essentially” is intended to mean that the recurring unit of formula I represents at least 95% of the (co)polymer according to the invention.

The (co)polymer according to the invention can consist mainly of the recurring unit of formula I. It is then a copolymer. The term “mainly” is intended to mean that the recurring unit of formula I represents at least 50% of the (co)polymer according to the invention.

The (co)polymer according to the invention can comprise the recurring unit of formula I in the minority. It is then a copolymer. The term “in the minority” is intended to mean that the recurring unit of formula I represents less than 50% of the (co)polymer according to the invention.

The (co)polymers obtained from a single diamine B or from a single diisocyanate B′ and from a single compound A are homopolymers. The (co)polymers obtained from a single diamine B or from a single diisocyanate B′ and from a single compound D are homopolymers.

The reaction between at least three different monomers produces a copolymer. The (co)polymers may be defined by the molar composition of each constituent monomer.

The (Co)Polymers

In formula I, the dashed bonds signify that the ring is either furanic (two double bonds) or tetrahydrofuranic (two single bonds). In other words, formula I corresponds to one of formula I.1 or I.2 below:

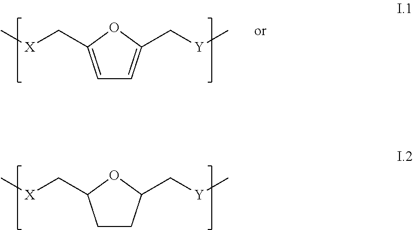

with X and Y corresponding to the definitions given above and below in the description.

In formula V of the polyamic acid according to the invention, the dashed bonds also signify that the ring is either furanic (two double bonds) or tetrahydrofuranic (two single bonds).

In formula I, R is a trivalent hydrocarbon-based group selected from:
a saturated or unsaturated, aliphatic or cycloaliphatic group, optionally interrupted with one or more heteroatoms, and optionally substituted with one or more functional or non-functional groups;
an aromatic, alkylaromatic or arylaliphatic group, optionally interrupted with one or more heteroatoms, and optionally substituted with one or more functional or non-functional groups.

The saturated or unsaturated, aliphatic or cycloaliphatic group advantageously has between 1 and 18 carbon atoms.

The aromatic, alkylaromatic or aryaliphatic group advantageously has between 4 and 18 carbon atoms.

The expression “optionally interrupted with one or more heteroatoms” is intended to mean that the hydrocarbon-based group can be interrupted with one or more following heteroatoms: O, N, S or P.

The expression “substituted with one or more functional or non-functional groups” is intended to mean that the hydrocarbon-based group can be substituted with following functional groups: —OH, C═O, sulfonate, sulfonic, —COOH, halogen (—F, —Cl, —Br); or with non-functional groups such as alkyls.

In formula I, R′ is a tetravalent hydrocarbon-based group selected from:
a saturated or unsaturated, aliphatic or cycloaliphatic group, optionally interrupted with one or more heteroatoms, and optionally substituted with one or more functional or non-functional groups;
an aromatic, alkylaromatic or arylaliphatic group, optionally interrupted with one or more heteroatoms, and optionally substituted with one or more functional or non-functional groups.

The saturated or unsaturated, aliphatic or cycloaliphatic group advantageously has between 1 and 18 carbon atoms.

The aromatic, alkylaromatic or aryaliphatic group advantageously has between 4 and 18 carbon atoms.

The expression “optionally interrupted with one or more heteroatoms” is intended to mean that the hydrocarbon-based group can be interrupted with one or more following heteroatoms: O, N, S or P.

The expression “substituted with one or more functional or non-functional groups” is intended to mean that the hydrocarbon-based group can be substituted with following functional groups: —OH, C═O, sulfonate, sulfonic, —COOH, halogen (—F, —Cl, —Br); or with non-functional groups such as alkyls.

The (co)polymers of the invention advantageously have a glass transition temperature Tg above 150° C., in particular above 200° C.

When a polymer has a Tg above 150° C., this signifies that, when the articles made with this polymer are used at a temperature below 150° C., the polymer is in its vitreous state, that is to say the state in which it is the most rigid. There are a large number of applications in which the temperature at which these articles are used does not exceed 150° C. and more particularly 200° C.: for example, a motor vehicle passenger compartment, buildings, packaging, electrics, electronics, etc. The proportioning of a part is carried out at the temperature at which the article will be used. Thus, if a semi-crystalline polymer of which the Tg is higher than the use temperature is used, the calculations take into account the high rigidity of the polymer. With respect to a semi-crystalline polymer of which the Tg would be below the use temperature, it can therefore be permitted, where appropriate, to use less material.

Quite particularly, the (co)polymers of the invention have a Tg below or equal to 300° C. This can in particular enable easier processing.

The (co)polymers according to the invention may be semi-crystalline or amorphous. If they are semi-crystalline, the (co)polymers according to the invention may thus have a melting point Mp ranging from 100 to 350° C., in particular from 150 to 350° C.

Generally, when the (co)polymer according to the invention is a (co)polyamideimide, that is to say it comprises at least one imide function and at least one amide function in its recurring unit I (when X=Ia and Y=Ic or Id), then it is a thermoplastic polymer, which can therefore be converted into articles via the melt process according to the processes conventionally used for thermoplastic polymers such as PA66.

When the (co)polymer according to the invention is a (co)polyimide, that is to say comprises two imide functions in its recurring unit I (when X=Ib and Y is a covalent bond), then it is generally not convertible via the melt process.

The amorphous (co)polymers have the advantage of being transparent (when they are not formulated), which is important in optics. In order to be able to be used, these polymers must imperatively have a Tg above the use temperature.

The semi-crystalline (co)polymers have the advantage of preserving mechanical properties above their Tg, up to their Mp or their degradation temperature.

The (co)polymers according to the invention advantageously have a number-average molar mass Mn ranging from 1000 to 100 000 g/mol, preferentially from 2000 to 50 000 g/mol and even more preferentially from 5000 to 30 000 g/mol.

The (co)polymers of the invention can have an excellent dimensional stability, that is to say they have a low linear thermal expansion coefficient LTEC, in particular which is similar to that of Kapton®.

Monomers

The Compounds A

One of the constituent monomers of the (co)polymer according to the invention can be at least one compound A comprising two anhydride functions and/or its tetracarboxylic acid and/or ester equivalents.

For the purposes of the present invention, the term “equivalents” or “derivatives” will be understood to mean that the “tetracarboxylic acid equivalents or derivatives” are hydrolyzed dianhydrides. For the purposes of the present invention, for the “ester equivalents or derivatives” it will be understood that they are the esters of the tetracarboxylic acid equivalents/derivatives, one or more acid functions being esterified with an alcohol.

The compounds A preferentially bear carboxylic acid functions in positions such that they enable the formation of two acid anhydride functions on one and the same molecule (by a dehydration reaction). The compounds A generally have two pairs of carboxylic acid functions, each function pair being bonded to an adjacent carbon atom, in the α and β positions.

The four equivalent carboxylic acid functions can be obtained from acid dianhydrides by hydrolysis of the anhydride functions. Examples of acid dianhydrides and of tetracarboxylic acids, derived from dianhydrides, are described in patent U.S. Pat. No. 7,932,012.

The compounds A of the invention can also bear at least one other functional group. This group can in particular be selected from the group —SO₃X, with X═H or a cation, in particular Na, Li, Zn, Ag, Ca, Al, K and Mg, the hydroxyl group —OH, the ketone group C═O, and ethers —O—.

The compounds A comprising two anhydride functions are preferentially selected from the group consisting of: ethane-1,1,2,2-tetracarboxylic dianhydride, butane-1,2,3,4-tetracarboxylic dianhydride, pentane-1,2,4,5-tetracarboxylic dianhydride, cyclobutane-1,2,3,4-tetracarboxylic dianhydride, cyclopentane-1,2,3,4-tetracarboxylic dianhydride, cyclohexane-1,2,4,5 tetracarboxylic dianhydride, cyclohexane-2,3,5,6-tetracarboxylic dianhydride, 3-ethyl-cyclohexane-3-(1,2)5,6-tetracarboxylic dianhydride, 1-methyl-3-ethyl-cyclohexane-3-(1,2)5,6-tetracarboxylic dianhydride, 1-ethyl-cyclohexane-1-(1,2),3,4-tetracarboxylic dianhydride, 1-propyl-cyclohexane-1-(2,3),3,4-tetracarboxylic dianhydride, 1,3-dipropyl-cyclohexane-1-(2,3),3-(2,3)-tetracarboxylic dianhydride, dicyclohexyl-3,4,3′,4′-tetracarboxylic dianhydride, tetrahydrofuran-2,3,4,5-tetracarboxylic dianhydride, pyromellitic anhydride, 3,3′,4,4′-biphenyltetracarboxylic dianhydride, 2,3,3′,4′-biphenyltetracarboxylic dianhydride, 2,2′,3,3′-biphenyltetracarboxylic dianhydride, 3,3′,4,4′-benzophenonetetracarboxylic dianhydride, 2,2′,3,3′-benzophenonetetracarboxylic dianhydride, 1,2,5,6-naphthalenetetracarboxylic dianhydride, 2,3,6,7-naphthalenetetracarboxylic dianhydride, 2,2′-bis(3,4-dicarboxyphenyl)hexafluoropropanetetracarboxylic dianhydride, 4,4′-oxydiphthalic dianhydride, 2,2-bis(3,4-dicarboxyphenol)sulfone dianhydride, bisphenol A dianhydride, 4,4′-(hexafluoroisopropylidene)diphthalic anhydride (6FDA), 3,4,9,10-perylenetetracarboxylic dianhydride, 3,3′,4,4′-diphenylsulfonetetracarboxylic dianhydride, 2,3,6,7-naphthalenetetracarboxylic dianhydride, ethylene glycol bis(trimellitic anhydride), hydroquinonediphthalic anhydride, 2,2-bis(3,4-dicarboxyphenyl)propane dianhydride, 1,1-bis(2,3-dicarboxyphenyl)ethane dianhydride, 1,1-bis(3,4-dicarboxyphenyl)ethane dianhydride, bis(2,3-dicarboxyphenyl)methane dianhydride, bis(3,4-dicarboxyphenyl)methane dianhydride, bis(3,4-dicarboxyphenyl)sulfoxide dianhydride, pyrazine-2,3,5,6-tetracarboxylic dianhydride, thiophene-2,3,4,5-tetracarboxylic dianhydride, phenanthrene-1,8,9,10-tetracarboxylic dianhydride, and mixtures thereof.

The compounds A comprising two anhydride functions that are preferred are selected from pyromellitic anhydride, 3,3′,4,4′-biphenyltetracarboxylic dianhydride, 4,4′-oxydiphthalic dianhydride, 3,3′,4,4′-benzophenonetetracarboxylic dianhydride, 2,2′,3,3′-benzophenonetetracarboxylic dianhydride, ethane-1,1,2,2-tetracarboxylic dianhydride, butane-1,2,3,4-tetracarboxylic dianhydride, and mixtures thereof. These compounds, in combination with the diamines B or the diisocyanates B′ according to the invention, can have the advantage of giving (co)polymers of which the Tg is above 150° C.

Among the dianhydrides mentioned above, pyromellitic dianhydride is particularly advantageous, in particular since it is inexpensive, it makes it possible to obtain polyimides with a very high Tg, it is widely available and it is easy to synthesize.

When the compound A is a tetracarboxylic acid, in particular derived from a compound comprising two anhydride functions, it is preferentially selected from the group consisting of: 1,2,3,4-butanetetracarboxylic acid, ethane-1,1,2,2-tetracarboxylic acid, pentane-1,2,4,5-tetracarboxylic acid, 1,2,3,4-cyclobutanetetracarboxylic acid, cyclopentane-1,2,3,4-tetracarboxylic acid, cyclohexane-1,2,4,5 tetracarboxylic acid, cyclohexane-2,3,5,6-tetracarboxylic acid, 3-ethyl-cyclohexane-3-(1,2)5,6-tetracarboxylic acid, 1-methyl-3-ethyl-cyclohexane-3-(1,2)5,6-tetracarboxylic acid, 1-ethyl-cyclohexane-1-(1,2),3,4-tetracarboxylic acid, 1-propyl-cyclohexane-1-(2,3),3,4-tetracarboxylic acid, 1,3-dipropyl-cyclohexane-1-(2,3),3-(2,3)-tetracarboxylic acid, dicyclohexyl-3,4,3′,4′-tetracarboxylic acid, tetrahydrofuran-2,3,4,5-tetracarboxylic acid, pyromellitic acid, 3,3′,4,4′-biphenyltetracarboxylic acid, 2,3,3′,4′-biphenyltetracarboxylic acid, 2,2%3,3′-biphenyltetracarboxylic acid, 3,3,4,4′-benzophenonetetracarboxylic acid, 2,2′,3,3′-benzophenonetetracarboxylic acid, 1,2,5,6-naphthalenetetracarboxylic acid, 2,3,6,7-naphthalenetetracarboxylic acid, 2,3,5,6-pyridinetetracarboxylic acid, 3,4,9,10-perylenetetracarboxylic acid, 3,3′,4,4′-tetraphenylsilanetetracarboxylic acid, 2,2′-bis(3,4-bicarboxyphenyl)hexafluoropropanetetracarboxylic acid, 4,4′-oxydiphthalic acid, 2,2-bis(3,4-dicarboxyphenol)sulfone acid, 4,4′-(hexafluoroisopropylidene)diphthalic acid, 3,4,9,10-perylenetetracarboxylic acid, 3,3′,4,4′-diphenylsulfonetetracarboxylic acid, 2,3,6,7-naphthalenetetracarboxylic acid, ethylene glycol bistrimellitic acid, hydroquinonediphthalic acid, 2,2-bis(3,4-dicarboxyphenyl)propane, 1,1-bis(2,3-dicarboxyphenyl)ethane, 1,1-bis(3,4-dicarboxyphenyl)ethane, bis(2,3-dicarboxyphenyl)methane, bis(3,4-dicarboxyphenyl)methane, bis(3,4-dicarboxyphenyl)sulfoxide, pyrazine-2,3,5,6-tetracarboxylic acid, thiophene-2,3,4,5-tetracarboxylic acid, phenanthrene-1,8,9,10-tetracarboxylic acid, and mixtures thereof.

Advantageously, when the compound A is a tetracarboxylic acid, in particular derived from a compound comprising two anhydride functions, it is preferentially selected from the group consisting of pyromellitic acid, 3,3′,4,4′-biphenyltetracarboxylic acid, 4,4′-oxydiphthalic acid, 3,3′,4,4′-benzophenonetetracarboxylic acid, 2,2′,3,3′-benzophenonetetracarboxylic acid, ethane-1,1,2,2-tetracarboxylic acid, butane-1,2,3,4-tetracarboxylic acid, and mixtures thereof.

Among the tetraacids (or tetracarboxylic acids) mentioned above, pyromellitic acid is particularly advantageous.

Alternatively, the compounds A of the invention may be the esters of the dianhydrides or of the tetraacids obtained by reaction of the dianhydride or of the tetraacid with a monoalcohol, such as methanol, ethanol, propanol and isomers, butanol, and mixtures thereof. They may be triacid monoesters, diacid diesters (or hemiesters), monoacid triesters, or tetraesters. Diacid diesters, and in particular the diester of pyromellitic acid, obtained by alcoholysis of pyromellitic anhydride with two alcohol molecules, are preferred.

In one particularly preferred embodiment of the invention, the compounds A are dianhydrides or tetracarboxylic acids since these compounds have the advantage of not giving off reaction by-products other than water, and in particular they do not give off solvents such as alcohols. In particular, pyromellitic anhydride or pyromellitic acid is preferred.

Advantageously, these preferred compounds A (pyromellitic anhydride or pyromellitic acid) represent at least 80 mol % relative to all the compounds A used.

Certain compounds A can have the advantage of being biobased, for instance butane-1,2,3,4-tetracarboxylic acid.

The Compounds D

The compounds D are advantageously selected from:
monoacid anhydrides such as trimellitic anhydride,
tricarboxylic acids such as trimellitic acid, tricarballylic acid, citric acid, aconitic acid, 1,2,4-butanetricarboxylic acid or 1,2,3-benzentricarboxylic acid,
diacid monoesters, monoacid diesters or triesters of the tricarboxylic acids above,
acid chloride anhydrides such as trimellitic anhydride chloride.

The Diamines B or the Diisocyanates B′

According to the invention, the term “or” used between the terms “diamine(s) B” and “diisocyanate(s) B′” signifies that the diamines B are not used in combination with the diisocyanates B′. The diisocyanates B′ are an alternative to the diamines B.

The diamines B according to the invention are selected from 2,5-bis(aminomethyl)furan and 2,5-bis(aminomethyl)tetrahydrofuran.

The diisocyanates B′ according to the invention are selected from the compounds of formulae II′ and III′.

In the particular embodiment according to which the diamine B is 2,5-bis-(aminomethyl)tetrahydrofuran or the diisocyanate B′ is the molecule of formula III′, it may be a question of the cis or trans stereoisomer or of a mixture thereof. For the cis stereoisomer, the chiral carbons in positions 2 and 5 may be R,S or S,R or a meso mixture. For the trans stereoisomer, the chiral carbons in positions 2 and 5 may be S,S or R,R or the racemic mixture.

These advantageously biobased diamines B may be synthesized, for example for 2,5-bis(aminomethyl)furan, by nitrilation of 2,5-furandicarboxylic acid followed by a selective hydrogenation, and a hydrogenation of the furan ring of 2,5-bis(aminomethyl)furan to prepare 2,5-bis(aminomethyl)tetrahydrofuran.

These advantageously biobased diisocyanates B′ may be synthesized for example, for the molecule of formula III′, by reaction of 2,5-bis(aminomethyl)tetrahydrofuran with phosgene according to the processes known for converting diamines to diisocyanate.

The Diamines C or the Diisocyanates C′

According to the invention, the term “or” used between the terms “diamine(s) C” and “diisocyanate(s) C′” signifies that the diamines C are not used in combination with the diisocyanates C′. The diisocyanates C′ are an alternative to the diamines C.

Likewise, according to the invention, the diisocyanates C′ are not used when the diamine B is used and, conversely, the diamines C are not used when the diisocyanates B′ are used.

In formula IV or IV′ below:

NH₂—R₃—NH₂ (IV)

O═C═N—R₃—N═C═O (IV′)
R₃ is a hydrocarbon-based divalent radical which is aliphatic, cycloaliphatic or arylaliphatic, and saturated and/or unsaturated, aromatic or alkylaromatic, and which optionally comprises heteroatoms.

The radical R₃ generally comprises from 2 to 100 carbon atoms, preferably from 4 to 50 carbon atoms. The radical R₃ may optionally contain one or more heteroatoms, such as O, N, P, or S. The radical R₃ may comprise one or more functional groups, such as hydroxyl, sulfone, ketone, ether, secondary amine, tertiary amine or other functions.

The diamines C or the diisocyanates C′ may in particular be diamines/diisocyanates in positions α and ω, containing from 4 to 20 methylene groups.

The aliphatic diamines C may, for example, be selected from the group consisting of: 1,2-diaminoethane, 1,3-diaminopropane, 1,4-diaminobutane, 1,5-diaminopentane, hexamethylenediamine, 2,2,4- and 2,4,4-trimethylhexamethylenediamine, 1,7-diaminoheptane, 1,8-diaminooctane, 2-methyl-1,8-diaminooctane, 2,2,7,7-tetramethyloctamethylenediamine, 1,9-diaminonane, 5-methyl-1,9-diaminononane, 1,10-diaminodecane, 1,11-diaminoundecane, 1,12-diaminododecane, 1,13-diaminotridecane, 1,14-diaminotetradecane, diamines derived from C36 fatty acid dimers, known for example under the name Priamine™ (reference 1075) sold by Croda.

The cycloaliphatic diamines C are selected, for example, from the group consisting of isophoronediamine, 1,3-bis(aminomethyl)cyclohexane, 1,4-bis(aminomethyl)cyclohexane, 1,3-diaminocyclohexane, 1,4-diaminocyclohexane and diaminodicyclohexylmethane.

The arylaliphatic diamines are, for example, meta-xylylenediamine and para-xylylenediamine.

Mention may be made of examples of diamines C containing heteroatoms, for instance polyether diamines, such as Jeffamine® and Elastamine® products sold by Huntsman. A variety of polyethers exist, composed of ethylene oxide, propylene oxide or tetramethylene oxide units. The number-average molar mass Mn is between 100 and 5000 g/mol.

The aromatic diamines C are, for example, selected from the following group: diaminodiphenylmethane and isomers thereof, sulfonyldianiline and isomers thereof, 3,4′-oxydianiline; 1,3-bis-(4-aminophenoxy)benzene; 1,3-bis(3-aminophenoxy)benzene; 4,4′-oxydianiline; 1,4-diaminobenzene; 1,3-diaminobenzene; 1,2-diaminobenzene, 2,2′-bis(trifluoromethyl)benzidene; 4,4′-diaminobiphenyl; 4,4′-diaminodiphenyl sulfide; 9,9′-bis(4-amino)fluorene; 4,4′-diaminodiphenylpropane; 4,4′-diaminodiphenylmethane; benzidine; 3,3′-dichlorobenzidine; 3,3′-diaminodiphenyl sulfone; 4,4′-diaminodiphenyl sulfone; 1,5-diaminonaphthalene; 4,4′-diaminodiphenyldiethylsilane; 4,4′-diaminodiphenysilane; 4,4′-diaminodiphenylethylphosphine oxide; 4,4′-diaminodiphenyl-N-methylamine and 4,4′-diaminodiphenyl-N-phenylamine.

Advantageously, the diamine C is selected from: meta-phenylenediamine, para-phenylenediamine, hexamethylenediamine, 2-methylpentamethylenediamine, 1,10-diaminodecane, 1,12-diaminododecane, meta-xylylenediamine, diaminodiphenylmethane and isomers thereof, and sulfonyldianiline and isomers thereof.

The diisocyanates C′ are advantageously selected from: isophorone diisocyanate (IPDI), hexamethylene diisocyanate (HDI), toluene diisocyanate (TDI) and methylene diphenyl diisocyanate (MDI), preferentially the 2,2′, 2,4′ and 4,4′ isomers.

Advantageously, when a mixture of diamines B and C is used, the diamine B represents at least 60 mol % relative to all the diamines B and C used, preferably at least 80 mol %.

Advantageously, when a mixture of diisocyanates B′ and C′ is used, the diisocyanate B′ represents at least 60 mol % relative to all the diisocyanates B′ and C′ used, preferably at least 80 mol %.

Particular Modes with the Diamines B and C

According to one particular embodiment of the invention, the (co)polymer according to the invention is obtained by polymerization of pyromellitic anhydride and 2,5-bis(aminomethyl)tetrahydrofuran. The polyimide thus obtained is particularly advantageous since it has a very high Tg, capable of rivalling non-biobased polyimides having a cycloaliphatic structure.

According to a second embodiment of the invention, the (co)polymer according to the invention is obtained by polymerization of trimellitic anhydride and 2,5-bis(aminomethyl)tetrahydrofuran. The polyamideimide thus obtained is particularly advantageous since it has a very high Tg and is thermoplastic.

According to a third embodiment of the invention, the (co)polymer according to the invention is obtained by polymerization of pyromellitic acid, 2,5-bis(aminomethyl)tetrahydrofuran and hexamethylene-1,6-diamine.

According to a fourth embodiment of the invention, the (co)polymer according to the invention is obtained by polymerization of pyromellitic acid, 2,5-bis(aminomethyl)tetrahydrofuran and 1,10-decanediamine.

According to another embodiment of the invention, the (co)polymer according to the invention is obtained by polymerization of 3,3′,4,4′-benzophenonetetracarboxylic dianhydride and 2,5-bis(aminomethyl)tetrahydrofuran.

Particular Modes with the Diisocyanates B′ and C′

According to one particular embodiment of the invention, the (co)polymer according to the invention is obtained by polymerization of trimellitic anhydride, the compound of formula III′ and isophorone diisocyanate.

According to another particular embodiment of the invention, the (co)polymer according to the invention is obtained by polymerization of trimellitic anhydride, the compound of formula III′ and MDI.

According to another particular embodiment of the invention, the (co)polymer according to the invention is obtained by polymerization of trimellitic anhydride, the compound of formula III′ and TDI.

The Salts

The present invention also relates to an ammonium carboxylate salt obtained from one or more compounds A (or D) and one or more diamines B and optionally one or more diamines C, said compounds A (or D), B and C being as defined above.

The salt according to the invention can also comprise at least one chain-limiting compound selected from monoamines, monoacids or diacids in the α,β positions such that they can form an anhydride function by dehydration reaction.

Advantageously, the chain-limiting compound is selected from: 1-aminopentane, 1-aminohexane, 1-aminoheptane, 1-aminooctane, 1-aminononane, 1-aminodecane, 1-aminoundecane, 1-aminododecane, benzylamine, ortho-phthalic acid (or 1,2-benzenedicarboxylic acid), acetic acid, propionic acid, benzoic acid, stearic acid or mixtures thereof.

Preference is given to 1-aminohexane, 1-aminododecane, benzylamine or mixtures thereof. These compounds have the effect of limiting the molar mass of the (co)polymer and thus of limiting the melt viscosity of the (co)polymer, thereby making it more readily transformable by remelting in order to produce articles.

The content of chain limiter may be from 0.1% to 10% by number of moles, in particular from 1% to 5% by number of moles, relative to the total number of moles of monomers, that is to say tetracarboxylic acid, diamine, and chain limiter.

Synthesis Processes

Several processes for producing the (co)polymers according to the invention by polymerization of the monomers A (or D), and optionally C or A (or D) and B′, and optionally C′, are possible, making it possible to obtain the (co)polymer in various forms (thermoplastic or non-thermoplastic powder, part obtained from the cyclization of a precursor of the polyamic acid type, etc.).

It is, for example, possible to carry out a solution (or solvent) polymerization, in particular by following the conventional routes for synthesizing polyimides in solvent, in 2 steps, for example proceeding via a polyamic acid (PAM).

A melt polymerization or solid polymerization of mixtures of monomers or using salts of these monomers can also be carried out.

Preferably, synthesis using polyamic acids, which enables the production of polyimide parts by means of heating a polyamic acid solution at a temperature below the decomposition temperature of the polyimide, will be chosen.

Synthesis in Solution (Proceeding Via PAM), Starting from the Diamines and Dianhydrides (Dianhydride Compounds A)

The process for synthesis of the (co)polymers via the solvent route is a process which comprises the following steps:
at least one compound A comprising two anhydride functions is reacted, at low temperature, between −20 and 120° C., preferentially between 20 and 80° C., in a polar aprotic solvent or a phenolic solvent, with a diamine B, and optionally in the presence of at least one diamine C, said compounds A, B and C being as previously defined, so as to form an intermediate referred to as polyamic acid. The reaction time can be between 5 min and 100 hours, preferably between 10 min and 10 hours;
heating is carried out at a temperature of between 120° C. and 350° C., preferentially between 140° C. and 300° C., and/or a chemical dehydration is carried out using, for example, acetic anhydride;
the (co)polymer is recovered, according to its solubility in the solvent, either by evaporation of the solvent in the case where the polymer is soluble in the solvent, or by precipitation from a non-solvent.

Preferably, the polar aprotic solvent is selected from dimethylacetamide, dimethylformamide, cresols or else N-methylpyrrolidone.

Preferably, the phenolic solvent is selected from phenol, 4-methoxyphenol, 2,6-dimethylphenol and m-cresol.

During the first step, the amines open the anhydride rings and give rise to an acid amide function, often called amic acid function. The polyamic acid formed is generally soluble in the synthesis solvent and is converted by cyclization to (co)polymer, which is usually insoluble.

Control of the number-average molar mass can be obtained:
through the use of chain limiters, that is to say molecules selected from monoamines, monoanhydrides, monoacids or diacids in the α, β positions such that they are able to form an anhydride function by dehydration reaction; among chain limiters, mention may be made of phthalic anhydride, 1-aminopentane, 1-aminohexane, 1-aminoheptane, 1-aminooctane, 1-aminononane, 1-aminodecane, 1-aminoundecane, 1-aminododecane, benzylamine, ortho-phthalic acid (or 1,2-benzenedicarboxylic acid), acetic acid, propionic acid, benzoic acid, stearic acid, or mixtures thereof,
through a stoichiometric imbalance r=[compounds A]/[diamines B+C],
through the use of branching agents, that is to say molecules with a functionality of more than 3,
through adjustment of the operating conditions for syntheses, such as the residence time, the temperature, the humidity or the pressure, or
through a combination of these various means.

The stoichiometry can be controlled at any point in the production process.

In particular, the stoichiometric imbalance r may range from 0.8 to 1.2.

The content of chain limiter may be from 0.1% to 10% by number of moles, in particular from 1% to 5% by number of moles, relative to the total number of moles of monomers, that is to say tetracarboxylic dianhydride, diamine, and chain limiter.

In this process, catalysts, inert or reactive inorganic fillers (clays, silicas or silica precursors, nanoparticles, etc.), stabilizers, matting agents or dyes can also be introduced.

For example, to make a (co)polymer film, it is possible to pour a solution of polyamic acid onto a heating surface. When the heating surface is heated, the solvent evaporates and cyclization takes place allowing a (co)polymer film to be obtained.

The (co)polymer may be soluble or insoluble in the solvent. If the (co)polymer is not soluble in the solvent, the (co)polymer can be obtained by heating the polyamic acid solution and precipitating. It can thus be recovered by filtration and drying: a powder is obtained. If the (co)polymer is soluble in the solvent, it can be recovered in powder form by precipitation from or with a non-solvent.

Examples of polymerizations according to the polyamic acid route are described in patent US 2009/0263640.

Synthesis in Solution, Starting from the Diisocyanates and the Dianhydrides A or the Diisocyanates of the Carboxylic Acid Anhydrides D

The process for synthesis of the (co)polymers via the solvent route is a process such that:
at least one compound A comprising two anhydride functions (or at least one compound D comprising an anhydride function and a carboxylic acid function) is reacted at a temperature ranging from 20 to 200° C., preferentially from 50 to 150° C., in a polar aprotic solvent, with a diisocyanate B′, and optionally in the presence of at least one diisocyanate C′, said compounds A (or D), B′ and C′ being as previously defined, so as to directly form the polyimide (or polyamideimide) in a single step. The reaction time can be between 5 min and 100 hours, preferably between 10 min and 10 hours. The reaction is monitored via the release of CO₂.

Synthesis Via the Molten Route

The syntheses via the molten route imply that the monomers or precursors are brought to a temperature:
above the melting point of the (co)polymer if the (co)polymer is semi-crystalline, or
above the glass transition temperature if the (co)polymer is amorphous.

The melt polymerization can be carried out starting from:
a diamine (B+optionally C) or a diisocyanate (B′+optionally C′) and a dianhydride or its tetraacid or diester or triester or tetraester derivatives (compound A), or a compound D,
a salt of diamine or a tetraacid or a diester.

Advantageously, the polymerization via the molten route is carried out starting from the salts, which has the advantage of precisely controlling the stoichiometry. In the case of the salts, an amount of water which allows dissolution of the salts or dispersion thereof can be added.

The reaction can be carried out in a synthesis reactor or in an extruder provided with a system for venting the vapors.

Melt polymerizations are in particular described in U.S. Pat. No. 2,710,853 starting from aliphatic diamine and pyromellitic anhydride or diacid diester derivatives of pyromellitic anhydride.

Control of the number-average molar mass may be obtained:
through the use of chain limiters, that is to say molecules selected from monoamines, monoanhydrides, monoacids or diacids in the α, β positions such that they are able to form an anhydride function by dehydration reaction; among chain limiters, mention may be made of phthalic anhydride, 1-aminopentane, 1-aminohexane, 1-aminoheptane, 1-aminooctane, 1-aminononane, 1-aminodecane, 1-aminoundecane, 1-aminododecane, benzylamine, ortho-phthalic acid (or 1,2-benzenedicarboxylic acid), acetic acid, propionic acid, benzoic acid, stearic acid, or mixtures thereof,
through a stoichiometric imbalance r=[compounds A]/[diamines B+C or diisocyanates B′+C′], or r=[compounds D]/[diamines B+C or diisocyanates B′+C′],
through the use of branching agents, that is to say molecules with a functionality of more than 3,
through adjustment of the operating conditions for syntheses, such as the residence time, the temperature, the humidity or the pressure, or
through a combination of these various means.

The stoichiometry can be controlled at any point in the production process.

In particular, the stoichiometric imbalance r may range from 1.01 to 1.2. This range is advantageous since it makes it possible to avoid the formation of gels by cross-linking of the amine.

The content of chain limiter may be from 0.1% to 10% by number of moles, in particular from 1% to 5% by number of moles, relative to the total number of moles of monomers, that is to say tetracarboxylic acid/dianhydride (or acid anhydride in the case of compound D), diamine/diisocyanate (B and optionally C or B′ and optionally C′) and chain limiter.

In this process, catalysts, inert or reactive inorganic fillers (clays, silicas or silica precursors, nanoparticles, etc.), stabilizers, matting agents or dyes can also be introduced.

Synthesis Via the Solid Route, Starting from the Tetracarboxylic Acids and Diamines or Tricarboxylic Acids and Diamines

The principle of the synthesis via the solid route consists in preparing the (co)polymer at a temperature below the melting point of the (co)polymer starting from a precursor; T<Mp(PI) in the case of a semi-crystalline (co)polymer or T<Tg(PI) in the case of an amorphous (co)polymer.

A novel route for industrial and efficient preparation of (co)polymers has just been demonstrated by the applicant.

This synthesis is made possible by the use of a solid-state polymerization of a solid salt of ammonium carboxylate formed from a diamine B and from a tetracarboxylic acid A, or of a solid salt of ammonium carboxylate formed from a diamine B and from a tricarboxylic acid D. The process of the invention produces powders of controlled particle size, since the polymerization reaction takes place in the solid state.

Furthermore, the solid-state polymerization avoids the use of carcinogenic or environmentally detrimental solvents.

Another advantage of the process of the invention is the capacity to carry out polymerization at a relatively low temperature, preventing thermal degradation of the salt and of the (co)polymer formed.

The present invention thus relates to a process for preparing (co)polyimides according to the invention, comprising at least the following steps:
(a) a salt formed by a reaction between at least one diamine B and at least one tetracarboxylic acid A or at least one diamine B and at least one tricarboxylic acid D is placed in a reactor;
(b) a solid-state polymerization is carried out starting from the salt of step (a) in order to obtain the (co)polymer at an absolute pressure of between 0.005 and 1 MPa and at a temperature T which obeys the following relationship, for a semi-crystalline (co)polymer:

Mp of the (co)polymer to be obtained>T

preferentially
Mp of the (co)polymer to be obtained>T>Tg of the (co)polymer to be obtained and even more preferentially
   Mp of the salt from step (a)>T>Tg of the (co)polymer to be obtained;
or at a temperature T which obeys the relationship T<Tg for an amorphous (co)polymer

and
(c) the solid (co)polymer particles are recovered.

Step (a)

During step (a) of the process, a salt formed by a reaction between at least one diamine B+/−C and at least one tetracarboxylic acid A or at least one diamine B+/−C and at least one tricarboxylic acid D is thus placed in a reactor.

Such a salt may be synthesized in various ways known to those skilled in the art.

One possible procedure, for example, is to add a diamine B+/−C to a solution comprising the tetracarboxylic acid A (or comprising the tricarboxylic acid D). Another possibility is to dissolve the tetracarboxylic acid A (or the tricarboxylic acid D) in a solvent such as alcohol, such as for example ethanol or methanol, and to do likewise for the diamine B+/−C. These two solutions are then mixed with stirring. The ammonium carboxylate salt formed may be insoluble in the solvent used and thus precipitate out. The salt may then be recovered by filtration, washed and dried, and optionally ground.

It is also possible to make a solution of the ammonium carboxylate salt and then to concentrate it while hot and then cool it. The salt then crystallizes and the crystals are recovered and dried. Concentration of the solution can be obtained by evaporation of the solvent such as water or alcohol or according to another process by addition of tetracarboxylic acid A (or tricarboxylic acid D) and/or of diamine B+/−C. It is also possible to perform saturation of the solution, i.e. to perform a process for modifying the concentration of the salt in the solution to a value that is compatible with its crystallization. Generally, this concentration is at least equal to and more preferentially greater than the saturation concentration of the salt at the temperature under consideration. More precisely, this concentration corresponds to supersaturation of the salt solution. It is also possible to work at a pressure that enables the solvent of the solution, such as the water or alcohol, to evaporate off, so as to saturate the solution and bring about crystallization. A further possibility is to saturate the solution by successive or simultaneous addition of a stream of tetracarboxylic acid A (or tricarboxylic acid D) and a stream of diamine B+/−C to a salt solution.

As an example, the tetracarboxylic acid is dissolved in the alcohol, such as ethanol, for example, in a first medium. The diamine B+/−C is dissolved in alcohol in another medium, and the two media are then mixed with stirring. The salt obtained precipitates out.

At the end of this synthesis, the salt may be in the form of a dry powder, in the form of a powder dispersed in a solvent, or dissolved in solution. The salt may be recovered by filtration in the case of a precipitate, and the filter cake may be disintegrated, if necessary. When the salt is dissolved in solution, it may be recovered via a crystallization process by concentration or supersaturation or by making it precipitate out by addition of a non-solvent. The crystallized salt may then be recovered by filtration and the filter cake may be disintegrated, if necessary. Another process for recovering the dispersed particles of dry salt is spraying of the solution, i.e. in particular an operation of sudden evaporation of the solvent sprayed in the form of fine droplets so as to recover the dispersed salt particles.

Finally, it is possible to screen the salt particle size, for example by sifting or milling.

Step (b)

During step (b) of the process, accordingly, a solid-state polymerization is carried out, starting from the salt from step (a), to give the (co)polymer, at an absolute pressure of between 0.005 and 1 MPa and at a temperature T which obeys the relationship as previously described.

The absolute pressure during step (b) is preferably between 0.005 MPa and 0.2 MPa.

The temperature during step (b) is preferably between 100° C. and 300° C.

The process of solid-state polymerization may be carried out according to the conventional processes known to those skilled in the art. The fundamental principle of these processes is to bring the initial salt, under air or in an inert atmosphere or under vacuum, to a temperature which is lower than its melting point but sufficient to allow the polymerization reaction, generally a temperature greater than the glass transition temperature of the (co)polymer. Such a process may thus comprise, in brief:
a) heating of the product by conductive or convective diffusion or by radiation,
b) inertizing by application of vacuum, flushing with a neutral gas such as nitrogen, CO₂, or superheated steam, or application of a positive pressure,
c) removal of the condensation by-product by evaporation, then flushing with the carrier gas or concentration of the gas phase,
d) mechanical stirring or fluidization of the solid phase with the carrier gas or vibration may be desirable in order to improve the heat and mass transfers and also to prevent any risk of agglomeration of the divided solid.

It is preferred in step b) to use a means for keeping the (co)polymer salt particles in motion, in order to prevent aggregation of these particles. This may also be accomplished by mechanical stirring, such as by use of a stirrer, by rotation of the reactor, or by vibratory agitation, or by fluidization with a carrier gas.

The number-average molar mass Mn of the (co)polymers may be between 1000 g/mol to 100 000 g/mol.

Control of the number-average molar mass may be obtained:
through the use of chain limiters, that is to say molecules selected from monoamines, monoanhydrides, monoacids or diacids in the α,β positions such that they can form an anhydride function by dehydration reaction. Examples of chain limiters are phthalic anhydride, 1-aminopentane, 1-aminohexane, 1-aminoheptane, 1-aminooctane, 1-aminononane, 1-aminodecane, 1-aminoundecane, 1-aminododecane, benzylamine, ortho-phthalic acid (or 1,2-benzenedicarboxylic acid), acetic acid, propionic acid, benzoic acid, stearic acid or mixtures thereof,
through a stoichiometric imbalance r=[compounds A]/[diamines B+/−C] or r=[compounds D]/[diamine B+/−C],
through the use of branching agents, that is to say molecules with a functionality of more than 3,
through adjustment of the operating conditions for syntheses, such as the residence time, the temperature, the humidity or the pressure, or
by a combination of these various means.

Control of the stoichiometry may be performed at any point in the manufacturing process.

In particular, the stoichiometric imbalance r may range from 1.01 to 1.2.

According to one particular embodiment:
chain limiters are added to the salt and/or
an excess of one of the monomers is added to the salt, in order to create a stoichiometric imbalance, that is to say in order for r to be different than 1.

According to one variant, the chain limiter is added to the salt of step (a) already formed.

According to another variant, the chain limiter is also in salt form, in particular it forms a salt with the diamine B+/−C and/or with the tetracarboxylic acid A (or the tricarboxylic acid D). In particular, the chain limiter is present during the formation of the salt of step (a) and is added at the same time as the species corresponding thereto, for example limiter of acid type with the tetracarboxylic acid A and limiter of amine type with the amine B+/−C.

In this second case, the chain limiter allows the formation of salt, and may be selected in particular from the above lists, with the exception of the anhydrides.

The content of chain limiter may be from 0.1% to 10% by number of moles, in particular from 1% to 5% by number of moles, relative to the total number of moles of monomers, that is to say tetracarboxylic acid A (or the tricarboxylic acid D), diamine B+/−C, and chain limiter.

When a chain limiter is used, the amounts of amines and of acids may be equilibrated, i.e., the sum of the amine functions is substantially equal to half the sum of acid functions with which they may react. The term “substantially equal” is intended to mean a maximum difference of 1%.

When a chain limiter is used, the amounts of amines and of acids may be imbalanced, i.e., the sum of the amine functions is substantially different from half the sum of acid functions with which they may react. The term “substantially different” is intended to mean a difference of at least 1%.

Catalysts can also be introduced, and also inert or reactive inorganic fillers (clays, silicas or silica precursors, nanoparticles, etc.), stabilizers, matting agents, dyes. etc.

Use may be made of catalysts, added at any point during the process, for instance as a mixture with the diamine and/or the tetracarboxylic acid, as a mixture with the salt formed, either in solution or by impregnation in the solid state.

Furthermore, applications exist for which the polymers are required to be in the form of powders. This is the case, in particular, with laser sintering or with processes for manufacture of continuous fiber composites from powders by dusting of fabrics or pultrusion of carbon or glass monofilament, or else other processes. The known technologies for producing polymer powders require either dissolving a polymer in a solvent and precipitating from a non-solvent—but this involves the use of toxic and carcinogenic solvents—or mixing the polymer in the melt state with an immiscible species, so as to generate segregation of the desired polymer, or else milling granules of formulated polymers, which imposes additional steps of micronization and drying. Whatever the case cited, the processes are complex and expensive.

Compositions

The Case of Thermoplastic (Co)Polymers:

The (co)polymer of the invention may be used to make compositions that are generally obtained by mixing of the various compounds, fillers and/or additives. The process is performed at more or less high temperature and at more or less high shear force, according to the nature of the various compounds. The compounds can be introduced simultaneously or successively. Use is generally made of an extrusion mixing device in which the material is heated, then melted and subjected to a shear force, and conveyed. According to particular embodiments, it is possible to prepare preblends, optionally in the melt state, before preparation of the final composition. It is possible, for example, to prepare a preblend in a resin, of the (co)polymer, for example, so as to produce a masterbatch.

The invention thus also relates to a process for producing a composition based on (co)polymers according to the invention, by melt or nonmelt mixing of said (co)polymer with reinforcing or bulking fillers and/or impact modifiers and/or additives.

The composition according to the invention can optionally comprise one or more other polymers, for instance polyamides, polyesters or polyolefins. These other polymers advantageously represent at least 40% by weight relative to the weight of the composition.

The composition of the invention may comprise between 20% and 90% by weight, preferably between 20% and 70% by weight, and more preferably between 35% and 65% by weight of (co)polymers according to the invention obtained by the polymerization process as previously described, relative to the total weight of the composition.

The composition can additionally comprise reinforcing or bulking fillers. Reinforcing or bulking fillers are fillers conventionally used for the production of thermoplastic compositions, in particular based on polyamide. Mention may in particular be made of reinforcing fibrous fillers, such as glass fibers, carbon fibers or organic fibers, non-fibrous fillers such as particulate or lamellar fillers and/or exfoliable or non-exfoliable nanofillers, for instance alumina, carbon black, clays, zirconium phosphate, kaolin, calcium carbonate, copper, diatomaceous earths, graphite, mica, silica, titanium dioxide, zeolites, talc, wollastonite, polymeric fillers, such as, for example, dimethacrylate particles, glass beads or glass powder. Preferably, in particular, reinforcing fibers, such as glass fibers, are used.

The composition according to the invention may comprise between 5% and 60% by weight of reinforcing or bulking fillers and preferentially between 10% and 40% by weight, relative to the total weight of the composition.

The composition of the invention comprising the (co)polymer as previously defined may comprise at least one impact modifier, that is to say a compound capable of modifying the impact strength of a (co)polymer composition. These impact modifier compounds preferentially comprise functional groups which react with the (co)polymer. According to the invention, the expression “functional groups that are reactive with the (co)polymer” is intended to mean groups that are capable of reacting or interacting chemically with the residual anhydride, acid, or amine functions of the (co)polymer, in particular by covalency, ionic or hydrogen-bond interaction, or van der Waals bonding. Such reactive groups make it possible to ensure good dispersing of the impact modifiers in the (co)polymer matrix. Examples include anhydride, epoxide, ester, amine and carboxylic acid functions and carboxylate or sulfonate derivatives.

The composition according to the invention may also comprise additives normally used for the manufacture of polyimide or polyamide compositions. Thus, mention may be made of lubricants, flame retardants, plasticizers, nucleating agents, anti-UV agents, catalysts, antioxidants, antistatic agents, dyes, matting agents, molding aids or other conventional additives.

These fillers, impact modifiers and/or additives may be added to the (co)polymers by suitable usual means that are well known in the field of engineering plastics, such as, for example, during salification, after salification, during the solid-state polymerization, or as a melt mixture.

The (co)polymer compositions are generally obtained by mixing the various compounds included in the composition under cold conditions or in the melt. The process is performed at more or less high temperature and at more or less high shear force, according to the nature of the various compounds. The compounds can be introduced simultaneously or successively. Use is generally made of an extrusion device in which the material is heated, then melted and subjected to a shear force, and conveyed.

It is possible to blend all the compounds in the melt phase during a single operation, for example during an extrusion operation. It is possible, for example, to blend granules of the polymer materials, to introduce them into the extrusion device in order to melt them and to subject them to more or less high shearing. According to specific embodiments, it is possible to preblend some of the compounds, in the melt or not in the melt, before preparation of the final composition.

The Case of Non-Thermoplastic (Co)Polymers (Proceeding Via Polyamic Acid)

In the case of the (co)polymers according to the invention which are not thermoplastic and cannot therefore be melted in order to be converted into articles, fillers can be added to the solid polyamic acid, in solution or dispersed in a solvent. These fillers may be reinforcing or bulking fillers and/or additives as mentioned above.

Applications

The Case of Thermoplastic (Co)Polymers:

The (co)polymer or the various compositions of the invention may be used for any shaping process for the manufacture of articles.

The invention thus also relates to a process for producing an article, using the (co)polymer according to the invention. To this end, mention may be made of various techniques such as the molding process, in particular injection molding, extrusion, extrusion blow-molding, or alternatively rotary molding, in particular in the field of motor vehicles, of electronics, of aeronautics and of electricity, for example. The extrusion process may in particular be a spinning process or a process for manufacturing films.

The present invention relates, for example, to the manufacture of articles of impregnated fabric type or composite articles containing continuous fibers. These articles may be manufactured, in particular, by contacting a fabric and (co)polymer particles of the invention in the solid or melt state. Fabrics are textile surfaces obtained by assembling yarns or fibers which are rendered integral by any process, in particular such as adhesive bonding, felting, braiding, weaving or knitting. These fabrics are also referred to as fibrous or filamentous networks, for example based on glass fiber, carbon fiber or the like. Their structure may be random, unidirectional (1D) or multidirectional (2D, 2.5D, 3D or other).

The (co)polymer particles of the invention may in particular be used in processes for manufacturing articles by selective melting of polymer powder layers—in particular, rapid prototyping by sintering in solid phase using a laser. Manufacture by selective melting of layers is a process for manufacturing articles that comprises laying down layers of materials in powder form, selectively melting a portion or a region of a layer, and laying down a new layer of powder, and again melting a portion of this layer, and so on, so as to give the desired object. The selectivity of the portion of the layer to be melted is obtained by means, for example, of the use of absorbers, inhibitors, or masks, or via the input of focused energy, for instance electromagnetic radiation such as a laser beam. Preference is given in particular to sintering by addition of layers, in particular to rapid prototyping by sintering using a laser.

The Case of Non-Thermoplastic (Co)Polymers (Proceeding Via Polyamic Acid):

In the case of the (co)polymers according to the invention which are not thermoplastic and cannot therefore be melted in order to be converted into articles, the potential applications are the production of films, of coatings, in particular in the electronics field, of molded parts, and of composite materials in combination with glass or carbon fibers. This may also involve fibers or filaments obtained by a process for example as described in

U.S. Pat. No. 4,460,526, comprising a step of dissolving the polyamic acid in a polar organic solvent, said solution then being subjected to a spinning step.

Properties

The (co)polymers of the invention have the advantage of being biobased, of having a very high Tg, of being very resistant, of being able to be semi-crystalline and of being able to provide hydrophilicity. Some may be thermoplastic and have the advantage of being melt-convertible. They also have good resistance to solvents of alcohol, xylene and NMP type.

A specific language is used in the description so as to facilitate understanding of the principle of the invention. Nevertheless, it should be understood that no limitation of the scope of the invention is envisaged by the use of this specific language.

The term “and/or” includes the meanings “and”, “or”, and all the other possible combinations of the elements connected to this term.

Other details or advantages of the invention will become more clearly apparent in the light of the examples given below purely by way of indication.

### EXPERIMENTAL SECTION

Measurement Standards:

The melting point (Mₚ) and the crystallization temperature on cooling (T_{c}) of the (co)polymers are determined by differential scanning calorimetry (DSC), using a Perkin Elmer Pyris 1 instrument, at a rate of 10° C./min. The Mₚ and T_{c} values of the (co)polymers are determined at the top of the melting and crystallization peaks. The glass transition temperature (Tg) is determined on the same instrument at a rate of 40° C./min (when possible, it is determined at 10° C./min and specified in the examples). The measurements are taken after melting the (co)polymer formed at T>(Mₚ of the (co)polymer+20° C.).

When polyimides are synthesized starting from salts, the melting point of the salt is determined as the end temperature of the endotherm measured by heating the salt at 10° C./min.

Thermogravimetric analysis (TGA) is carried out on a Perkin-Elmer TGA7 instrument on a sample of around 10 mg, by heating at 10° C./min with nitrogen flushing up to 600° C.

Proton NMR analysis is carried out on a Brüker AV500 spectrometer.

### EXAMPLE 1

### Synthesis of the PI TFPMA

The diamine 2,5-bis(aminomethyl)tetrahydrofuran (TF) cis/trans 90/10 is synthesized according to the following procedure:
The starting molecule, tetrahydrofuran-2,5-dimethanol, is synthesized from the reaction, in methanol, of 5-(hydroxymethyl)furfural (HMF) with Raney nickel (1.5 equivalents relative to the HMF) under a pressure of 5.84 bar of H₂ at 60° C. for 20 hours, filtration and purification by distillation. A slightly yellow liquid is obtained, with a purity greater than 98% (determined by chromatography/coupled to mass spectrometry). Reaction yield: 95%.
Methanesulfonyl chloride (307.8 g, 2.7 mol) is added dropwise to a solution of tetrahydrofuran-2,5-dimethanol (118.8 g, 900 mmol) and of triethylamine (454.5g, 4500 mmol) in 1.54 l of dichloromethane at 0° C. The reaction medium is maintained at 0° C. for 1 hour and then added to ice-cold water and the organic phase is separated and washed with 500 ml of a dilute hydrochloric acid solution (1M). The organic phase is separated and then washed with 500 ml of an aqueous solution saturated with NaHCO₃. The organic phase is finally separated and concentrated to give 236.7 g of (tetrahydrofuran-2,5-diyl)bis(methylene)dimethanesulfonate in the form of a brown oil having a purity equal to 96% (determined by LCMS). Reaction yield: 91.0%. A solution of (tetrahydrofuran-2,5-diyl)bis(methylene)dimethanesulfonate (236.7 g, 821.7 mmol) and NaN₃ (270.0 g, 4.1094 mol) in DMSO (1.350 l) is heated at 95° C. and stirred overnight. The reaction medium is added to ice-cold water and extracted with 3×700 ml of ethyl acetate. The extracts (phase containing the ethyl acetate) are successively washed with water and an aqueous solution saturated with NaHCO₃ and dried overnight over MgSO₄ and then filtered in order to remove the MgSO₄. The phase containing the ethyl acetate is concentrated to give 166.5 g of 2,5-bis(azidomethyl)tetrahydrofuran in the form of a brown oil. A mixture of 2,5-bis(azidomethyl)tetrahydrofuran (166.5 g) and Pd—C (10%, 10.8 g) in methanol (2.71) is stirred overnight at ambient temperature under 1 atm of H₂. The reaction medium is filtered and the filtrate is concentrated under vacuum to give 90.0 g of 2,5-bis(aminomethyl)tetrahydrofuran in the form of a yellow oil. The total yield from the 3 successive reactions is 75%. The process for synthesizing 2,5-bis(aminomethyl)tetrahydrofuran gives a 90/10 mixture of the cis/trans isomers according to the C¹³ NMR analysis in deuterated methanol.

A methanol solution (solution 1) is prepared in a flask by dissolving 1.78 g (0.0137 mol) of 2,5-bis(aminomethyl)tetrahydrofuran in 15 g of methanol at ambient temperature. 20 g of methanol and 3.39 g (0.0133) of pyromellitic acid (solution 2) are introduced into a 100 ml round-bottomed flask at ambient temperature, with stirring. When all the pyromellitic acid is dissolved, solution 1 is introduced into solution 2, with stirring, over the course of 2 minutes. The empty flask of solution 1 is rinsed with 10 g of methanol and again added to the round-bottomed flask. The reaction medium is stirred at ambient temperature for 2 hours.

The precipitate formed (TFPMA salt) is recovered by filtration, washed with methanol and dried. The proton NMR analysis in dimethyl sulfoxide shows that the salt is stoichiometric. The salt is placed in a glass tube rendered inert with a stream of nitrogen, and heated at 210° C. for 5 hours in order to carry out the polymerization. The salt powder is converted into polyimide PI TFPMA powder, with water being given off.

The DSC thermal analysis (first heating at 10° C./min) up to 300° C. shows an exothermic phenomenon with a peak at 263° C. associated with crystallization. After 1 minute at 300° C., the sample is cooled to 20° C. at 10° C./min, maintained at 20° C. for 1 min and heated to 300° C. at 10° C./min. A glass transition temperature Tg at 256° C. is thus observed, but melting is not observed (greater than 300° C.).

The PI TFPMA is therefore a semi-crystalline polyimide, with Mp>300° C. and with Tg=256° C. It has a Tg of the same order of magnitude as that of the polyimide PI 1,3-BAMCPMA obtained from pyromellitic acid and 1,3-bis(aminomethyl)cyclohexane described in patent JP2012092262 (Tg=260° C.). The two polymers are semi-crystalline and have a similar Tg; the PI-TFPMA is therefore clearly a biobased alternative to the PI 1,3-BAMCPMA.

### COMPARATIVE EXAMPLE

### Synthesis of the PI MXDPMA

The same procedure as that of example 1 is used to prepare a powder of MXDPMA salt from meta-xylylenediamine (1.762 g, i.e. 0.0129 mol) and pyromellitic acid (3.34 g, 0.0131 mol). The salt powder is recovered by filtration, washed, dried and heated at 210° C. for 5 hours in order to carry out the polymerization.

The Tg of the PI MXDPMA measured under the same conditions as those of example 1 is detected at 275° C.

Conclusion: the PI TFPMA provides an advantageous biobased alternative to the PI MXDPMA and PI 1,3-BAMCPMA.

This PI MXDPMA is soluble at ambient temperature at 10 g/l in 96% concentrated sulfuric acid and insoluble at ambient temperature at 10 g/l in formic acid or 1,3-dimethylimidazolidinone. The PI TFPMA exhibits the same behavior; it therefore provides, from the point of view of both the thermal properties and the chemical properties, a biobased alternative to polyimides derived from fossil resources.

1. A (co)polymer comprising the recurring unit of following formula I: 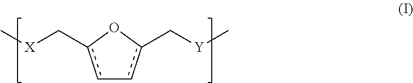
in which
X is a divalent group of formula Ia or Ib below: 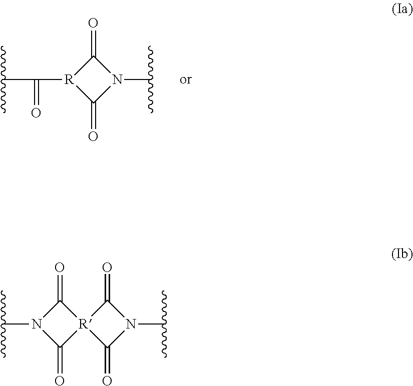
in which
R is a trivalent hydrocarbon-based group selected from:
a saturated or unsaturated, aliphatic or cycloaliphatic group, optionally interrupted with one or more heteroatoms, and optionally substituted with one or more functional or non-functional groups;
an aromatic, alkylaromatic or arylaliphatic group, optionally interrupted with one or more heteroatoms, and optionally substituted with one or more functional or non-functional groups;
R′ is a tetravalent hydrocarbon-based group selected from:
a saturated or unsaturated, aliphatic or cycloaliphatic group, optionally interrupted with one or more heteroatoms, and optionally substituted with one or more functional or non-functional groups;
an aromatic, alkylaromatic or arylaliphatic group, optionally interrupted with one or more heteroatoms, and optionally substituted with one or more functional or non-functional groups;
Y is:
a covalent bond, when X corresponds to formula Ib,
a divalent group of formula Ic or Id below, when X corresponds to formula Ia: 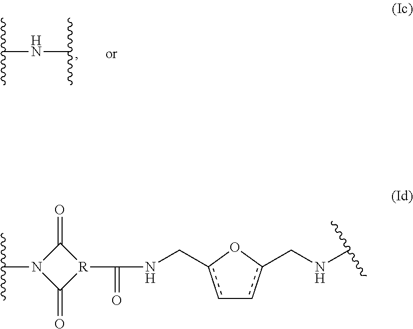
with R as defined above.

2. The (co)polymer as claimed in claim 1, wherein the (co)polymer is obtained by polymerization of the following compounds:
at least one compound A comprising two anhydride functions and/or its tetracarboxylic acid and/or ester equivalents, and
at least one diamine B of formula II or III, 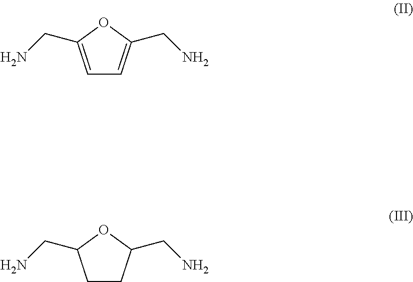 [SMILES: NCc1ccc(CN)o1 ; NCC1CCC(CN)O1]
or
at least one diisocyanate B′ of formula II′ or III′ below: 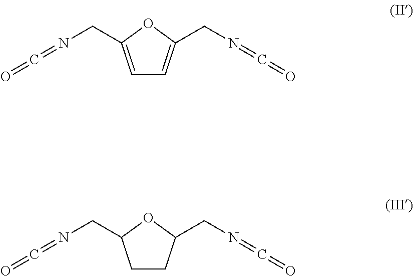 [SMILES: O=C=NCc1ccc(CN=C=O)o1 ; O=C=NCC1CCC(CN=C=O)O1]
or
at least one ammonium carboxylate salt obtained from the compounds A and B.

3. The (co)polymer as claimed in claim 2, wherein compound A is pyromellitic anhydride or pyromellitic acid.

4. The (co)polymer as claimed in claim 1, wherein the (co)polymer is obtained by polymerization of the following compounds:
at least one compound D comprising an anhydride function and a carboxylic acid function and/or its tricarboxylic acid and/or ester and/or acid chloride anhydride equivalents; and
at least one diamine B of formula II or III, 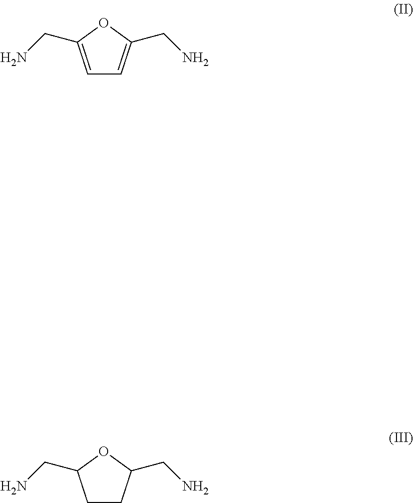 [SMILES: NCc1ccc(CN)o1 ; NCC1CCC(CN)O1]
or
at least one diisocyanate B′ of formula II′ or III′ below: 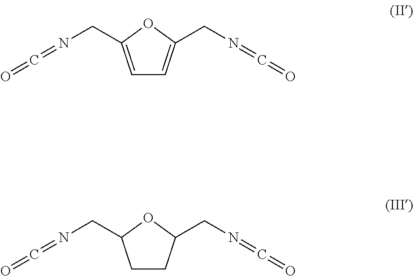 [SMILES: O=C=NCc1ccc(CN=C=O)o1 ; O=C=NCC1CCC(CN=C=O)O1]
or
at least one ammonium carboxylate salt obtained from the compounds D and B.

5. The (co)polymer as claimed in claim 4, wherein compound D is selected from:
trimellitic anhydride,
tricarboxylic acids selected from trimellitic acid, tricarballylic acid, citric acid, aconitic acid, 1,2,4-butanetricarboxylic acid and 1,2,3-benzentricarboxylic acid,
diacid monoesters, monoacid diesters or triesters of said tricarboxylic acids,
trimellitic anhydride chloride.

6. The (co)polymer as claimed in claim 2, wherein diamine B is a diamine of formula III below:

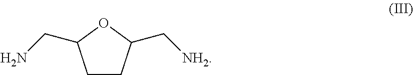

7. The (co)polymer as claimed in claim 1, wherein the (co)polymer is obtained by polymerization of at least the compound A or D and of at least the diamine B or of at least the diisocyanate B′, in the presence of at least one diamine C or of at least one diisocyanate C′ of formula IV or IV′ below:
NH₂—R₃—NH₂ (IV)
O═C═N—R₃—N═C═O (IV′)
with R₃ being a hydrocarbon-based divalent radical which is aliphatic, cycloaliphatic or arylaliphatic, and saturated and/or unsaturated, aromatic or alkylaromatic, and which optionally comprises heteroatoms.

8. A polyamic acid corresponding to formula V below: 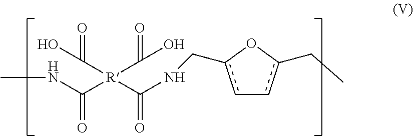
wherein R′ is a tetravalent hydrocarbon-based group selected from:
a saturated or unsaturated, aliphatic or cycloaliphatic group, optionally interrupted with one or more heteroatoms, and optionally substituted with one or more functional or non-functional groups;
an aromatic, alkylaromatic or arylaliphatic group, optionally interrupted with one or more heteroatoms, and optionally substituted with one or more functional or non-functional groups.

9. A process for producing a (co)polymer according to claim 1, the process comprising:
reacting at least one compound A comprising two anhydride functions is reacted with a diamine B of formula II or III, 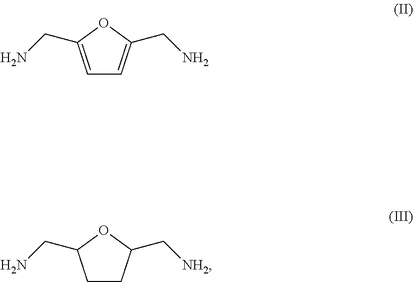 [SMILES: NCc1ccc(CN)o1 ; NCC1CCC(CN)O1]
between −20 and 120° C., in a polar aprotic solvent or a phenolic solvent, and optionally in the presence of at least one diamine C of formula IV:
NH₂—R₃—NH₂ (IV)
with R₃ being a hydrocarbon-based divalent radical which is aliphatic, cycloaliphatic or arylaliphatic, and saturated and/or unsaturated, aromatic or alkylaromatic, and which optionally comprises heteroatoms, so as to form a polyamic acid intermediate of formula V 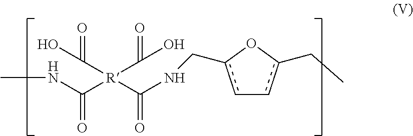
wherein R′ is a tetravalent hydrocarbon-based group selected from:
a saturated or unsaturated, aliphatic or cycloaliphatic group, optionally interrupted with one or more heteroatoms, and optionally substituted with one or more functional or non-functional groups;
an aromatic, alkylaromatic or arylaliphatic group, optionally interrupted with one or more heteroatoms, and optionally substituted with one or more functional or non-functional groups;
heating is carried out at a temperature of between 120° C. and 350° C. and/or carrying out a chemical dehydration;
recovering the (co)polymer either by evaporation of the solvent, or by precipitation from a non-solvent.

10. A process for producing a (co)polymer according to claim 1, the process comprising carrying out solution polymerization, solid-state polymerization or melt polymerization of at least one compound A and of at least one diamine B, optionally in the presence of at least one diamine C, or by solution polymerization of an ammonium carboxylate salt obtained from said compounds A, B and optionally C,
wherein compound A comprises two anhydride functions,
diamine B is of formula II or III, 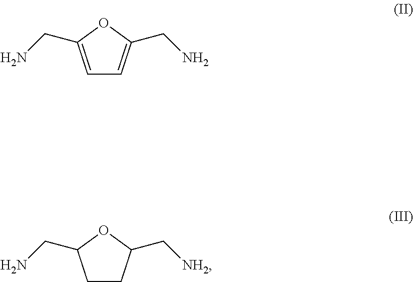 [SMILES: NCc1ccc(CN)o1 ; NCC1CCC(CN)O1]
and
diamine C is of formula IV:
NH₂—R₃—NH₂ (IV)
with R₃ being a hydrocarbon-based divalent radical which is aliphatic, cycloaliphatic or arylaliphatic, and saturated and/or unsaturated, aromatic or alkylaromatic, and which optionally comprises heteroatoms.

11. An ammonium carboxylate salt obtained from one or more compounds A or D and one or more diamines B and optionally one or more diamines C,
wherein compound A comprises two anhydride functions,
diamine B is of formula II or III, 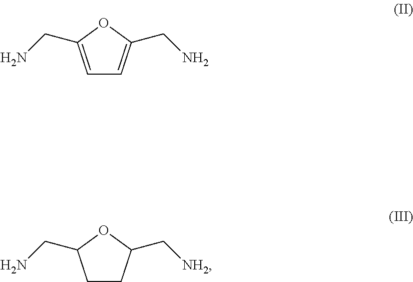 [SMILES: NCc1ccc(CN)o1 ; NCC1CCC(CN)O1]
and
diamine C is of formula IV:
NH₂—R₃—NH₂ (IV)
with R₃ being a hydrocarbon-based divalent radical which is aliphatic, cycloaliphatic or arylaliphatic, and saturated and/or unsaturated, aromatic or alkylaromatic, and which optionally comprises heteroatoms, and
compound D comprises an anhydride function and a carboxylic acid function.

12. A composition comprising the (co)polymer as defined in claim 1 and fillers and/or additives.

13. A process for producing an article based on (co)polymer as claimed in claim 1, the process comprising shaping by extrusion, molding or blow molding of said (co)polymer in molten form when it has a melting point below 350° C. for a semi-crystalline (co)polymer or a glass transition temperature below 300° C. for an amorphous (co)polymer.

14. A process for producing an article based on (co)polymer as claimed in claim 1, the process comprising cyclizing a polyamic acid, in solid form or in the form of a solution or a dispersion in a solvent
wherein the polyamic acid corresponds to formula V below: 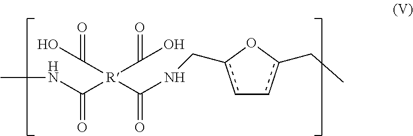
wherein R′ is a tetravalent hydrocarbon-based group selected from:
a saturated or unsaturated, aliphatic or cycloaliphatic group, optionally interrupted with one or more heteroatoms, and optionally substituted with one or more functional or non-functional groups;
an aromatic, alkylaromatic or arylaliphatic group, optionally interrupted with one or more heteroatoms, and optionally substituted with one or more functional or non-functional groups.

15. An article obtained from the (co)polymer as defined in claim 1.

16. The article as claimed in claim 15, wherein the article is a molded, extruded or blow-molded part.

17. The article as claimed in claim 16, wherein the article is an injection-molded part, a composite comprising continuous fibers, a film, a fiber, a yarn or a filament, a foam or a part woven or knitted from said fibers, yarns or filaments.

18. An article obtained from the composition as defined in claim 12.

19. The article as claimed in claim 18, wherein the article is a molded, extruded or blow-molded part.

20. The article as claimed in claim 19, wherein the article is an injection-molded part, a composite comprising continuous fibers, a film, a fiber, a yarn or a filament, a foam or a part woven or knitted from said fibers, yarns or filaments.
